(19) United States
(12) Patent Application Publication
(10) Pub. No.: US 2020/0008711 A1
(43) Pub. Date: Jan. 9, 2020

(54) AIRWAY SIZING APPARATUS

(71) Applicant: SPIRATION, INC., d/b/a OLYMPUS RESPIRATORY AMERICA, Redmond, WA (US)

(72) Inventors: Christine Jurevicius, Kent, WA (US); Jean-Martin Baillargeon, Seattle, WA (US)

(21) Appl. No.: 16/493,043

(22) PCT Filed: Mar. 17, 2017
(86) PCT No.: PCT/US2017/022930

§ 371 Date: Sep. 11, 2019

(51) Int. Cl. A61B 5/107; A61B 5/00; A61B 5/06

(52) U.S. Cl. CPC A61B 5/1076; A61B 5/065; A61B 5/6847

(57) ABSTRACT

An airway sizing apparatus (10) for determining a cross-sectional length (20) of an airway (30) of a subject (40) comprising: a guide rod assembly (50) including one or more control members (52) and a distal scale (54); a collar assembly (70) disposed on the guide rod assembly, configured to radially expand and contract with activation of the one or more control members; wherein the collar assembly includes a collar indicator member (72) that moves linearly on the distal scale to indicate the cross-sectional length of the airway.

### FIELD

The present teachings generally relate to an airway sizing apparatus for sizing a passageway for a mechanical airway valve.

### BACKGROUND

The present teachings are predicated upon providing an airway sizing apparatus that allows a user to measure a passageway and preferably an airway of a subject so that a valve can be selected for use in the airway. Currently, passageways are measured using balloons that are inflated using a non-compressible fluid such as saline until the balloon fills the passageway. During filling of the balloon air is bled from the balloon to ensure that a proper measurement and proper sealing by the valve will be achieved. Over inflation or under inflation of the balloon also are monitored during filling. The amount of fluid placed in the balloon during sizing is then compared to a sizing chart to determine the proper sized valve to seal a given airway. While this process is very effective at measuring passageways, the process may have to be repeated one or more times in order to ensure that a correctly sized valve is selected for a given passageway and bleeding the balloon may extend the procedure. An example of such a device and method is available from Spiration at http://www.spiration.com/sites/default/files/pagefiles/PI-03175AB_ASK_IFU-HUG_WEB.pdf last accessed on Feb. 15, 2016 the teachings of which are expressly incorporated by reference herein in their entirety for all purposes. An example of another device is sold under the name Aero Sizer by Merit Medical Endotek, the teachings of which are expressly incorporated by reference herein for all purposes.

Other passageways are measured using electronic sizing devices that are expanded into contact with a wall of a passageway and a digital read out is provided. An example of a sizing device is found in U.S. Pat. No. 8,357,139 the teachings of which are expressly incorporated by reference herein in their entirety for all purposes.

Other valve devices are conformable to the size of the airway such that the airway does not need to be measured and a one size fits all device may be employed. However, if the size of the device is not selected properly or the device is not set in the airway, the device may be expelled (e.g., coughed out) or may move within the passageway such that the effectiveness of the device is reduced. An example of such a device is found in U.S. Pat. No. 7,854,228 the teachings of which are expressly incorporated by reference herein in their entirety for all purposes.

Examples of other sizing device may be found in U.S. Patent Application Publication No. 2012/0149978; International Patent Application Publication No. WO2015/153493; and PCT/US17/15636, the teachings of which are all incorporated by reference herein in their entirety for all purposes.

It would be attractive to have a sizing device that accurately measures a cross-sectional length of a passageway. It would be attractive to have a sizing device that measures non-round passageways so that a valve can be selected that will seal the passageway and remain in place. What is needed is a planning tool that allows a user to measure both a cross-sectional length and potentially an axial length of a passageway at the same time without any repositioning of the airway sizing apparatus. What is needed is an airway sizing apparatus that indicates if a passageway is one size or larger than the airway sizing apparatus and/or one size or smaller than the airway sizing apparatus.

### SUMMARY

The present teachings meet one or more (if not all) of the present needs by providing an apparatus comprising:

The present teachings provide a kit comprising: two or more airway sizing apparatuses of the teachings herein.

A method comprising: (1) compacting the airway sizing apparatus of the teachings herein into a retracted state, and (2) inserting the airway sizing apparatus into a working channel of a bronchoscope.

The present teachings provide a sizing device that accurately measures a cross-sectional length of a passageway. The present teachings provide a planning tool that allows a user to measure a cross-sectional length of a passageway at the same time without any repositioning of the airway sizing apparatus. The present teachings provide an airway sizing apparatus that indicates if a discrete cross-sectional length value.

### BRIEF DESCRIPTION OF THE DRAWINGS

FIG. 1A illustrates a perspective view of one embodiment of the present invention in a bronchoscope.

FIG. 1B is a close up view of the distal end of FIG. 1A.

FIG. 2A is a perspective view of one embodiment of the present invention as deployed in a subject.

FIG. 2B is a close up view of the distal end of FIG. 2A

FIGS. 3A-C are perspective views of one embodiment of the present invention in-situ in an airway at three stages of deployment.

### DETAILED DESCRIPTION

The explanations and illustrations presented herein are intended to acquaint others skilled in the art with the invention, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The present teachings are directed to an improved airway sizing apparatus. The airway sizing apparatus functions to measure one or more cross-sectional lengths of a structure. The airway sizing apparatus may measure one or more diameters of a structure. Preferably, the structure is a passageway and more preferably the structure is an airway. Preferably, the airway sizing apparatus measures a plurality of cross-sectional lengths (e.g., diameter) of a passageway. More preferably, the airway sizing apparatus measures a passageway to determine which valve can seal the passageway. For example, the apparatus may measure a cross-sectional length of a passageway from one side to the other despite the passageway being non-circular or irregular in shape. If the passageway is circular then the cross-sectional length is a diameter. The airway sizing apparatus may function to measure an axial length of a passageway. The airway sizing apparatus may measure if there are any obstructions within a given axial length that would affect a valve from deploying, being retained in a location, sealing, or a combination thereof. Preferably, the airway sizing apparatus allows a user to simultaneously measure one or more cross-sectional lengths and one or more axial lengths of a passageway. The axial length is a length of the valve or airway sizing apparatus from a distal end to a proximal end. The axial length may be a distance that the valve spans when retracted, deployed, or both.

The airway sizing apparatus in a retracted state may fit within a delivery catheter, a sheath, or a channel of a bronchoscope. Preferably, the airway sizing apparatus in a retracted tool will fit within a bronchoscope. The airway sizing apparatus may have collar assembly that may include: a wire or series of wires that are expandable and retractable; a collar indicator member that moves linearly on a distal scale to indicate the cross-sectional length of the airway. The wire or series of wires, or portions thereof, may have dimensions that mirror those of a valve. The wire or series of wires may have an expanded diameter or cross-sectional length of about 5 mm or more, about 7 mm or more, about 10 mm or more, about 12 mm or more. The wire or series of wires may have an expanded diameter or cross-sectional length of about 20 mm or less, about 15 mm or less, or about 13 mm or less. The wire or series of wires of the airway sizing apparatus may be compressed (in a retracted state) to fit within the bronchoscope and may expand (in a deployed state) to substantially fill a length of a passageway such as an airway or a branch of bronchia or a bronchiole.

The deployed state may have the wires are partially or fully expanded, extending radially outward from the guide rod assembly (the guide rod assembly is essentially a hollow shaft that contains, houses, or provides support for the collar assembly). The deployed state may be so that the wires are substantially in contact with a wall of a passageway and a discrete cross-sectional length value of the airway may be indicated on the distal scale (to indicate the cross-sectional length of the airway). The airway sizing apparatus may be moved from the deployed state back into the retracted state so that the airway sizing apparatus may be removed. The airway sizing apparatus may be retracted from the proximal end to the distal end.

The proximal end may be an end of the airway sizing apparatus closest to a user, an opening of a passageway, or both. The proximal end may function as a handle and for location of user controls (controlling expanding and retracting the collar assembly). The largest cross-sectional length of the airway sizing apparatus may be located at the proximal end. The proximal end may include one or more proximal scales, graphical user interfaces, or communication devices such as electrical wires for a transducer (described below). The proximal end may be located opposite a distal end. The distal end may be located the farthest into a passageway. The distal end may include a portion of the collar assembly, one or more transducers, a distal scale. The distal end may extend beyond a location of where a distal end of a valve may extend. The distal end may extend to a location where the delivery catheter extends. A guide rod and control members may extend from the proximal end to the distal end.

The guide rod functions to carry the collar assembly so that a passageway can be measured. The guide rod may be a longitudinal axis of the airway sizing apparatus. The guide rod may be rigid. The guide rod may be flexible. The guide rod may be hollow. The guide rod may be made of plastic, metal, a bio-compatible material, or a combination thereof. The guide rod may have one or more components that extend through the guide rod, for example control members and/or locking members. The guide rod may be used to move (e.g., push) the airway sizing apparatus into place. The guide rod may be used to move (e.g., pull) the airway sizing apparatus out of the passageway. The guide rod may include one or more conduits that extend from the proximal end to the distal end. The guide rod may include one or more control members (e.g., wires, strings, cables) that may extend through a length of the guide rod. The guide rod may include one or more control members that extend through the guide rod and in communication with the collar assembly.

The collar assembly, or parts thereof, may function to measure a cross-sectional length of a passageway. Preferably, the collar assembly function to measure a cross-sectional length of a passageway in the desired sealing area in a passageway. More preferably, the collar assembly includes a wire (or set of wires) in conjunction with the distal scale that indicate or measures a cross-sectional length of a passageway at a sealing area at a single location. The wires are expandable and contractible. The wires may have a portion that moves along the length of the guide rod. The wires may be manipulated by a control member. The wires may be manipulated by an actuator device that extends through the guide rod. The wires may be slidably connected to the guide rod. The wires may be elastically deformable, for example to create a generally spherical shape when deployed. Preferably, the wires are not elastically deformable (i.e., the wires may only expand to a predetermined size). The wires may conform to non-round shapes. The wires may be made of a non-compliant material or a semi-compliant material. The wires in the deployed state, may be made of nitinol, stainless steel, polyethylene terephthalate, polyester, a thermoplastic, polypropylene, polyether, a polyether block amide, polyamide, polyester, polyurethane, a minimally-stretchable plastic, a minimally-stretchable biocompatible plastic, a non-elastic plastic, a non-stretchable plastic, or a combination thereof. The wires may be deployed using the control members. The wires may have a shape that is flat, spherical, egg shaped, one or more cones, one or more pyramids, one or more pentagons, diamond shape, oval, round shaped, kite shaped, two back to back pyramids, or a combination thereof. The wires, in the deployed state, may have a location along the length where the wires are farthest (radially) from the guide rod. The wires, in the deployed state, may gradually increase in distance from the guide rod, gradually decrease in distance from the guide rod, or both. The wires may have one or more segments that run parallel to the guide rod. The wires may be two back to back shapes. For example, the wires may be take the shape of two cones that are back to back with a linear segment connecting the cones. The linear segment may be a seal area indicator.

A distal scale may function as a visual indicator of the cross-sectional length of the airway. The scale may be a series of (3 or more) color bands, numbers, symbols, or any combination thereof that correspond to a discrete value (e.g. diameter) of the cross-sectional length of the airway. For example, a green band may indicate a 6 mm length, a red band a 7 mm length, a blue band a 8 mm length, and so on. Optionally, there may be a corresponding proximal scale disposed outside of the subject that functions in a similar fashion.

A collar indicator member that is slidably disposed at or near the distal end of the guide rod (as part of the collar assembly) functions to indicate the cross-sectional length of the airway via the distal scale. It may comprise a pointer to or a window over the distal scale or any number of constructions, so long as it provides the indication function. Optionally, there may be a corresponding proximal indicator member disposed outside of the subject that functions in a similar fashion.

A transducer (position sensor) at or near the distal area of the apparatus may function to produce an electronic signal that can correspond to a position of the collar indicator member or the wires which is correlated to the cross-sectional length of the airway. It is believed that any number of sensor configurations may be used to accomplish the desired function. A position sensor is any device that permits position measurement. It can either be an absolute position sensor or a relative one (displacement sensor). Position sensors can be linear, angular, or multi-axis. Examples of some position sensors available today are as follows: Capacitive transducer; Capacitive displacement sensor; Eddy-current sensor; Ultrasonic sensor; Grating sensor; Hall effect sensor; Inductive non-contact position sensors; Laser Doppler Vibrometer (optical); Linear variable differential transformer (LVDT); Multi-axis displacement transducer; Photodiode array; Piezo-electric transducer (piezo-electric); Potentiometer; Proximity sensor (optical); Rotary encoder (angular); Seismic displacement pick-up; and String potentiometer (also known as string pot., string encoder, cable position transducer). The transducer may communicate this position to some appropriate graphical user interface disposed outside the subject.

A seal area indicator on the wires may function to indicate a sealing location of a valve. The seal area indicator may function to align a delivery catheter with an area of interest. The airway sizing apparatus, the delivery catheter, or both may include a seal area indicator. The seal area indicator of the delivery catheter may ensure that the seal area of a valve aligns with the location measured by the seal area indicator of the airway sizing apparatus. The seal area indicator may measure a sealing location of a valve. The seal area indicator may have a length that is substantially the same as an area to be sealed by a valve. The cross-sectional length of the seal area indicator of the airway sizing apparatus may be substantially identical to the cross-sectional length of the valve (i.e., the difference may be about 1 mm or less, preferably about 0.5 mm or less, or more preferably about 0.25 mm or less). The seal area indicator of the airway sizing apparatus may have a maximum diameter. The seal area indicator of the airway sizing apparatus may comprise an area or portion of the wire that has a bright color, thus making it easier to view when the wire is deployed. The seal area indicator of the airway sizing apparatus may indicate how the valve will seal a non-round shape, an irregular shape, or both. The seal area indicator of the airway sizing apparatus may indicate how a range of cross-sectional lengths will seal. The seal area indicator may be at a proximal end of the seal area indicator, indicate a proximal end of a valve, or both. The seal area indicator of the airway sizing apparatus may indicate a maximum size (e.g., diameter) that a valve can seal. The seal area indicator of the airway sizing apparatus may have a largest cross-sectional length of about 4 mm or more, about 5 mm or more, about 6 mm or more, about 7 mm or more, or about 9 mm or more. The seal area indicator of the airway sizing apparatus may have a largest cross sectional length of about 12 mm or less or about 10 mm or less. The seal area indicator may be used in conjunction with a distal and/or proximal scale to determine the best size valve to use for a site of a passageway.

The passageway may be any passageway in a lung. The passageway may be a trachea, a bronchi, bronchiole, a branch of a bronchi, a branch of a bronchiole, or a combination thereof. The passageway may be any passageway that may be sealed by a valve to control airflow. The valve may include an anchor, a plurality of struts, a plurality of anchors, a membrane, a seal area, or a combination thereof. Examples, of exemplary valves that the present teachings may be used with are found in U.S. Pat. Nos. 8,647,392; 7,942,931; and 7,533,671 the teachings of with are expressly incorporated by reference herein for all purposes regarding a valve and its components. The anchor may hold the valve in place upon deployment. The struts may expand radially outward and contact a wall of a passageway. The struts may extend outward and open a membrane. The membrane may prevent airflow from extending past the valve. The valve and airway sizing apparatus may be included individually or together in a kit.

The kit may include one or more of the airway sizing apparatuses of the teachings herein. The kit may include one or more valves. The kit may include one airway sizing apparatus for each of the valves that may be used. The kit may include an inflation syringe. The kit may include a measuring device. The kit preferably includes two or more airway sizing apparatuses, three or more airway sizing apparatuses, and four or more airway sizing apparatuses. The kit may include an instructions manual. The kit may include one or more airway sizing apparatuses, two or more airway sizing apparatuses, three or more airway sizing apparatuses, or even four or more airway sizing apparatuses. When the kit includes more than one airway sizing apparatus the airway sizing apparatuses are all a different size. Preferably, the kit includes the same number of airway sizing apparatuses as there are available valve sizes.

The airway sizing apparatus may be used in a method that may employ one or more of the steps herein that may be employed in virtually any order. A method may be employed to size a passageway. The method may include a step of loading the airway sizing apparatus in a delivery catheter. The method may include a step of retracting the airway sizing apparatus. The method may include a step inserting the delivery catheter into a passageway. Retracting the delivery catheter. Moving the airway sizing apparatus into the passageway. Inflating the balloon of the airway sizing apparatus. Visually inspecting the distal scale, the proximal scale, the seal area indicator, or a combination thereof. The visual inspection may include moving the imaging device around the outside of the airway sizing apparatus to inspect the distal scale. The visual inspection may be performed during deployment of the wires. The visual inspection compares the airway sizing apparatus to the structure to determine if the valve can seal the structure (i.e., passageway). Determining the size of the valve. Removing the airway sizing apparatus. Inserting a second airway sizing apparatus or a third airway sizing apparatus into the passageway and repeating the steps taught herein.

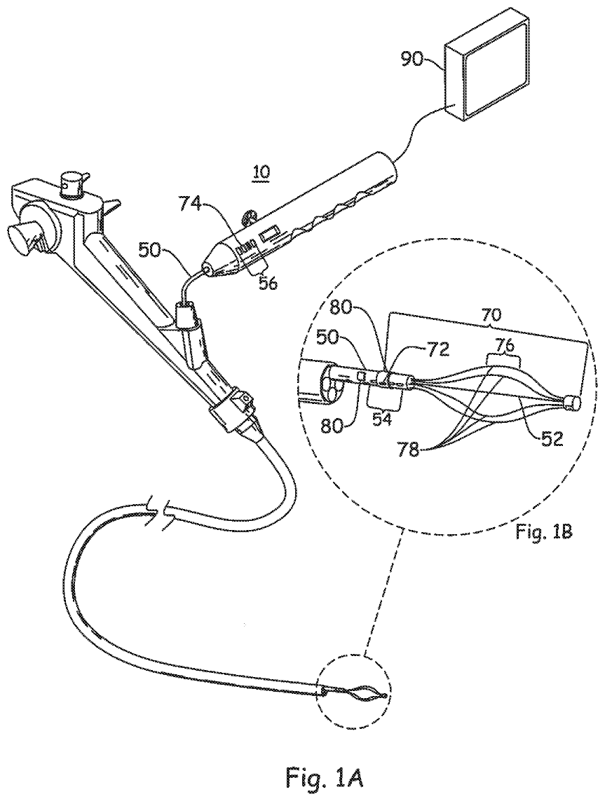

FIG. 1A illustrates a perspective view of one embodiment of the present invention in a bronchoscope. In this example, the apparatus 10 is shown in place, in the working channel of a bronchoscope. A graphical user interface (GUI 90) is also shown. FIG. 1B is a close up view of the distal end of FIG. 1A. In this figure, the distal end of the guide rod assembly 50, the collar assembly 70 (including the Collar indicator member 72 and deployed wires 78), the control members 52, a location of a transducer 80, and a distal scale 54 is shown.

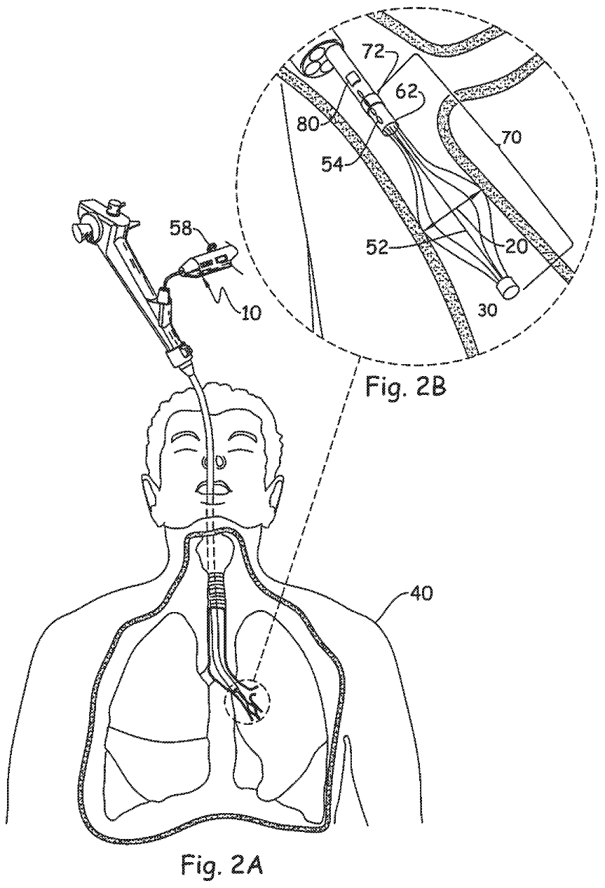

FIG. 2A is a perspective view of one embodiment of the present invention as deployed in an airway 30 of a subject 40. FIG. 2B is a close up view of the distal end of FIG. 2A. showing components of the apparatus and the airway cross-sectional length 20.

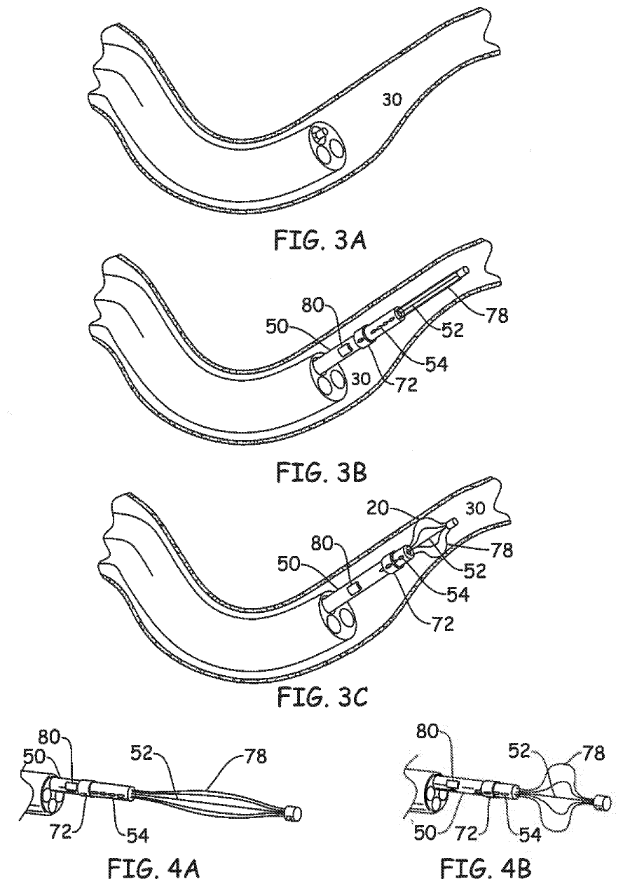

FIGS. 3A-C are perspective views of one embodiment of the present invention in-situ in an airway at three stages of deployment. In 3A, the apparatus 10 is immerging from the distal end of a bronchoscope. In 3B, a partially deployed apparatus 10 is shown and in 3C a fully deployed apparatus 10 can be seen.

### ELEMENT LIST

Airway sizing apparatus 10
Airway cross-sectional length 20
Airway 30
Subject 40
Guide rod assembly 50
Control members 52
Distal scale 54
Proximal scale 56
Locking member 58
Proximal end of guide rod 59
Distal end of guide rod 62
Collar assembly 70
Collar indicator member 72
proximal indicator members 74
seal area indicator 76
Wires 78
Transducer (position sensor) 80
GUI 90

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps. By use of the term “may” herein, it is intended that any described attributes that “may” be included are optional.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

1. An airway sizing apparatus for determining a cross-sectional length of an airway of a subject, the airway sizing comprising:
a guide rod assembly including one or more control members and a distal scale;
a collar assembly including one or more wires disposed on or in the guide rod assembly, the one or more wires configured to radially expand and contract with activation of the one or more control members;
wherein the collar assembly includes a collar indicator member that moves linearly on the distal scale to indicate the cross-sectional length of the airway.

2. The airway sizing apparatus of claim 1 further comprising:
the one or more control members including a locking member configured to allow the collar assembly to only radially expand and remain in a radially expanded position until the locking member is unlocked.

3. The airway sizing apparatus of claim 1 further comprising:
a transducer disposed near a distal end of the guide rod assembly, the transducer configured to produce an electronic signal corresponding to a position of the collar indicator member which is correlated to the cross-sectional length of the airway and communicate the position to a graphical user interface disposed outside the subject.

4. The airway sizing apparatus of claim 1 further comprising:
the distal scale comprising a color-coded scale with three or more colors, each color corresponding to a discrete cross-sectional length value of the airway.

5. The airway sizing apparatus of claim 1 further comprising:
a proximal scale disposed on the airway sizing apparatus, disposed outside of the subject when in use, and includes a corresponding proximal indicator member to indicate the cross-sectional length of the airway on the proximal scale.

6. The airway sizing apparatus of claim 1 further comprising:
wherein the one or more wires forming a spherical shape when the one or more control members are retracted towards a proximal end of the guide rod.

7. The airway sizing apparatus of claim 2 further comprising:
a transducer disposed near a distal end of the guide rod assembly, the transducer configured to produce an electronic signal corresponding to a position of the collar indicator member which is correlated to the cross-sectional length of the airway and communicate the position to a graphical user interface disposed outside the subject.

8. The airway sizing apparatus of claim 2 further comprising:
a proximal scale disposed on the airway sizing apparatus, disposed outside of the subject when in use, and includes a corresponding proximal indicator member to indicate the cross-sectional length of the airway on the proximal scale.

9. The airway sizing apparatus of claim 7 further comprising:
a proximal scale disposed on the airway sizing apparatus, disposed outside of the subject when in use, and includes a corresponding proximal indicator member to indicate the cross-sectional length of the airway on the proximal scale.

10. The airway sizing apparatus of claim 2 further comprising:
wherein the one or more wires forming a spherical shape when the one or more control members are retracted towards a proximal end of the guide rod.

11. The airway sizing apparatus of claim 9 further comprising:
wherein the one or more wires forming a spherical shape when the one or more control members are retracted towards a proximal end of the guide rod.
